(19) United States
(12) Patent Application Publication
(10) Pub. No.: US 2009/0149706 A1
(43) Pub. Date: Jun. 11, 2009

(54) ENDOSCOPE APPARATUS AND SIGNAL PROCESSING METHOD THEREOF

(75) Inventors: Kenji YAMAZAKI, Tokyo (JP); Kazuhiro GONO, Kanagawa (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/372,202

(22) Filed: Feb. 17, 2009

Related U.S. Application Data
(63) Continuation of application No. PCT/JP2007/058671, filed on Apr. 20, 2007.

(30) Foreign Application Priority Data
Aug. 18, 2006 (JP) 2006-223576

(51) Int. Cl. A61B 1/04

(52) U.S. Cl. 600/109

(57) ABSTRACT

A respective band signal conversion section of the present invention generates WLI-R, WL-G, and WLI-B for generating a normal observation light image and NBI-R, NBI-G, and NBI-B for generating a narrow-band light image from an RGB image signal obtained by irradiation with frame sequential light of a set of a rotary filter, and a synthesis circuit synthesizes frame sequential color signals of WLI-R, WLI-G, and WLI-B and frame sequential color signals of NBI-R, NBI-G, and NBI-B. Thus it is possible to simultaneously observe a same living tissue in real time in the normal light observation image and narrow-band light observation with a simple configuration.

### CROSS REFERENCE TO RELATED APPLICATION

This application is a continuation application of PCT/JP2007/058671 filed on Apr. 20, 2007 and claims benefit of Japanese Application No. 2006-223576 filed in Japan on Aug. 18, 2006, the contents of which are incorporated herein by this reference.

### BACKGROUND OF INVENTION

1. Field of the Invention

The present invention relates to an endoscope apparatus and particularly relates to an endoscope apparatus which picks up an image of a living tissue and performs signal processing, and a signal processing method of the apparatus.

2. Description of the Related Art

Conventionally, endoscope apparatuses have been widely used which apply illumination light to obtain endoscope images in body cavities. In such an endoscope apparatus, an electronic endoscope is used which has image pickup means for guiding illumination light into a body cavity from a light source device with a light guide and the like and picking up an image of a subject through return light. By performing signal processing on an image pickup signal from the image pickup means by a video processor, an endoscope image is displayed on an observation monitor to enable observation of an observation part such as a diseased part.

When a normal observation of a living tissue is performed in the endoscope apparatus, white light of a visible light region is emitted by the light source device. Frame sequential light is applied to a subject through, for example, an RGB rotary filter and the like, and return light obtained from the frame sequential light is synchronized and is subjected to image processing by the video processor, so that a color image is obtained. Alternatively, a color chip is placed at a front of an image pickup surface of the image pickup means in the endoscope, and the return light obtained from white light is separated into color components to pick up an image and the image is subjected to image processing by the video processor, so that a color image is obtained.

On a living tissue, light absorption characteristics and scattering characteristics vary with the wavelength of applied light. For example, Japanese Patent Application Laid-Open Publication No. 2002-95635 proposes a narrow-band light endoscope apparatus which emits illumination light of a visible light region, irradiates a living tissue with narrow-band RGB frame sequential light having discrete spectral characteristics, and obtains tissue information at a desired depth of the living tissue.

### SUMMARY OF THE INVENTION

An endoscope apparatus according to an aspect of the present invention includes:

an illuminating unit for applying illumination light to a subject;

a biological image information acquiring unit for receiving a subject image of the subject having been irradiated with the illumination light from the illuminating unit, and obtaining biological image information of the subject;

a band limiting unit which is disposed on an optical path from the illuminating unit to the biological image information acquiring unit and limits, to a predetermined bandwidth, at least one of a plurality of wavelength bands allocated according to penetration depths of light in the subject;

a biological image information converting section for converting the biological image information obtained by the biological image information acquiring unit, to first biological image signal information corresponding to irradiation with band limited light of the plurality of wavelength bands with the predetermined bandwidth and second biological image information corresponding to irradiation with the illumination light; and

a display image generating unit for generating a display image to be displayed on a display unit, based on the first biological image signal information and the second biological image signal information which have been converted by the biological image information converting section.

A signal processing method of an endoscope apparatus according to an aspect of the present invention includes:

an illuminating step of applying illumination light to a subject;

a biological image information acquiring step of receiving a subject image of the subject having been irradiated with the illumination light, and obtaining biological image information of the subject;

a band limiting step of limiting, to a predetermined bandwidth, at least one of a plurality of wavelength bands allocated according to penetration depths of light in the subject, on an optical path from the illuminating unit to the biological image information acquiring unit;

a biological image information converting step of converting the biological image information obtained in the biological image information acquiring step, to first biological image signal information corresponding to irradiation with band limited light of the plurality of wavelength bands with the predetermined bandwidth and second biological image information corresponding to irradiation with the illumination light; and

a display image generating step of generating a display image to be displayed on a display unit, based on the first biological image signal information and the second biological image signal information which have been converted in the biological image information converting step.

### BRIEF DESCRIPTION OF THE DRAWINGS

FIG. 1 is a structural diagram showing a configuration of an endoscope apparatus according to a first embodiment of the present invention;

FIG. 2 is a structural diagram showing a configuration of a rotary filter shown in FIG. 1;

FIG. 3 is a diagram showing spectral characteristics of a filter set of the rotary filter;

FIG. 4 is a structural diagram showing a configuration of a respective band signal conversion section shown in FIG. 1;

FIG. 5 is a diagram showing amplitude characteristics of a BPF shown in FIG. 4;

FIG. 6 is a first diagram showing a display example of an observation monitor shown in FIG. 1;

FIG. 7 is a second diagram showing a display example of the observation monitor shown in FIG. 1;

FIG. 8 is a third diagram showing a display example of the observation monitor shown in FIG. 1;

FIG. 9 is a diagram showing γ correction characteristics of a γ correction circuit shown in FIG. 1;

FIG. 10 is a structural diagram showing a configuration of an endoscope apparatus according to a second embodiment of the present invention;

FIG. 11 is a structural diagram showing a configuration of a primary color filter shown in FIG. 10;

FIG. 12 is a diagram showing a transmission property of the primary color filter shown in FIG. 11;

FIG. 13 is a structural diagram showing a configuration of a respective band signal conversion section shown in FIG. 10;

FIG. 14 is a structural diagram showing a configuration of an endoscope apparatus according to a third embodiment of the present invention;

FIG. 15 is a diagram showing a transmission property of a heat ray cut-off filter shown in FIG. 14;

FIG. 16 is a structural diagram showing a configuration of a complementary color filter shown in FIG. 14;

FIG. 17 is a structural diagram showing a configuration of a respective band signal conversion section shown in FIG. 14;

FIG. 18 is a diagram showing a modification of a transmission property of the heat ray cut-off filter shown in FIG. 14; and

FIG. 19 is a structural diagram showing a configuration of a modification of a respective band signal conversion section shown in FIG. 14.

### DETAILED DESCRIPTION OF THE PREFERRED EMBODIMENT(S)

Embodiments of the present invention will be described below in accordance with accompanying drawings.

### First Embodiment

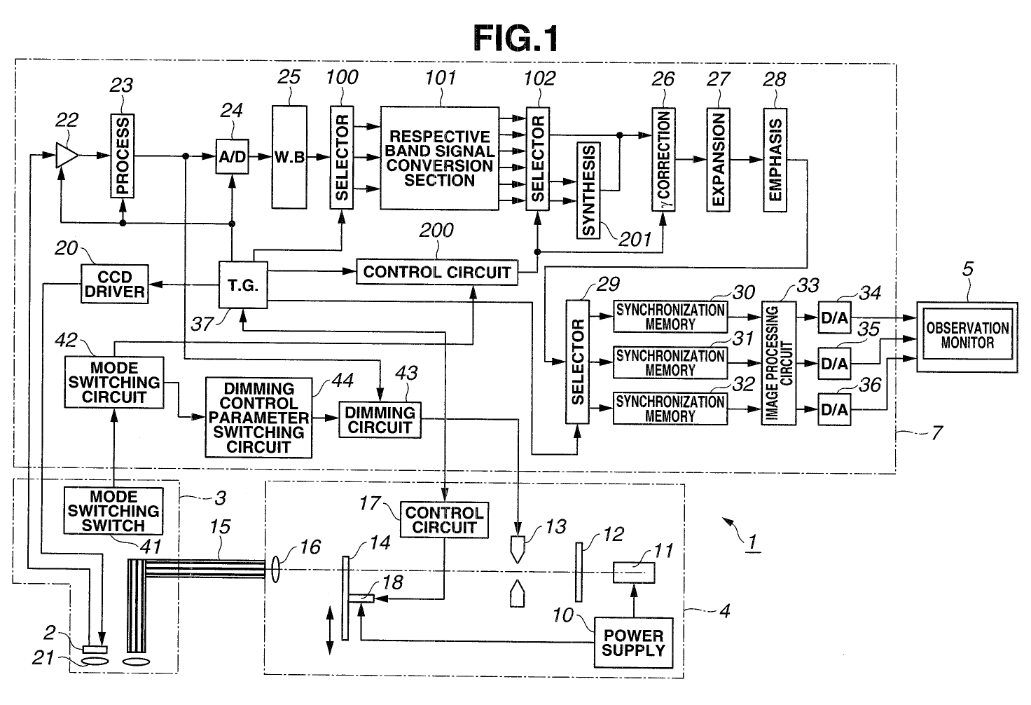

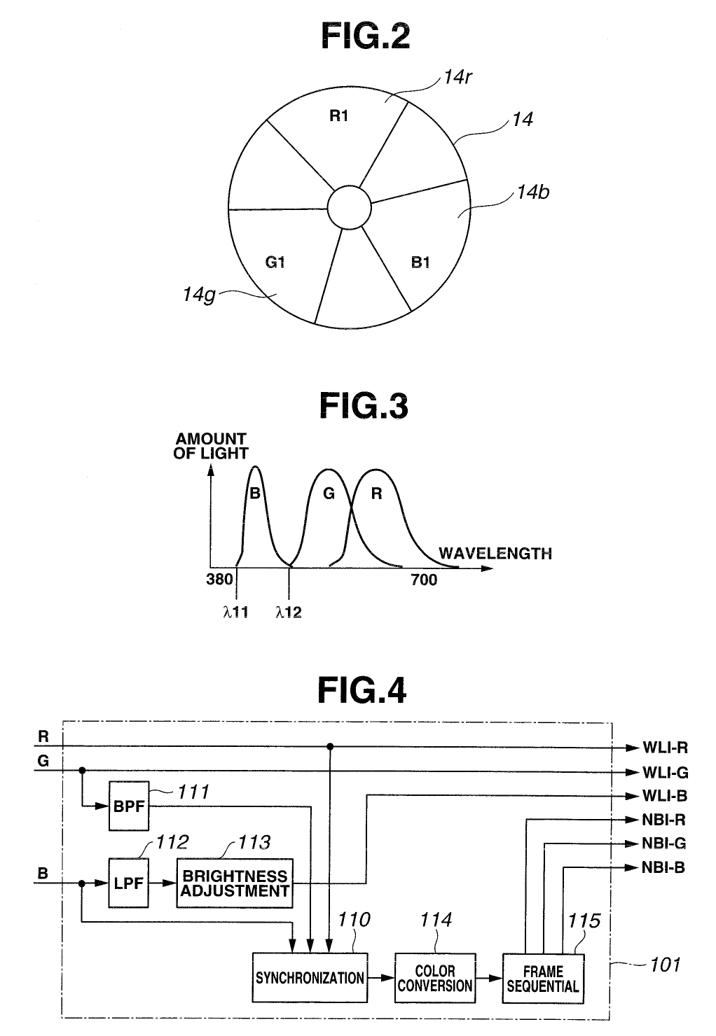

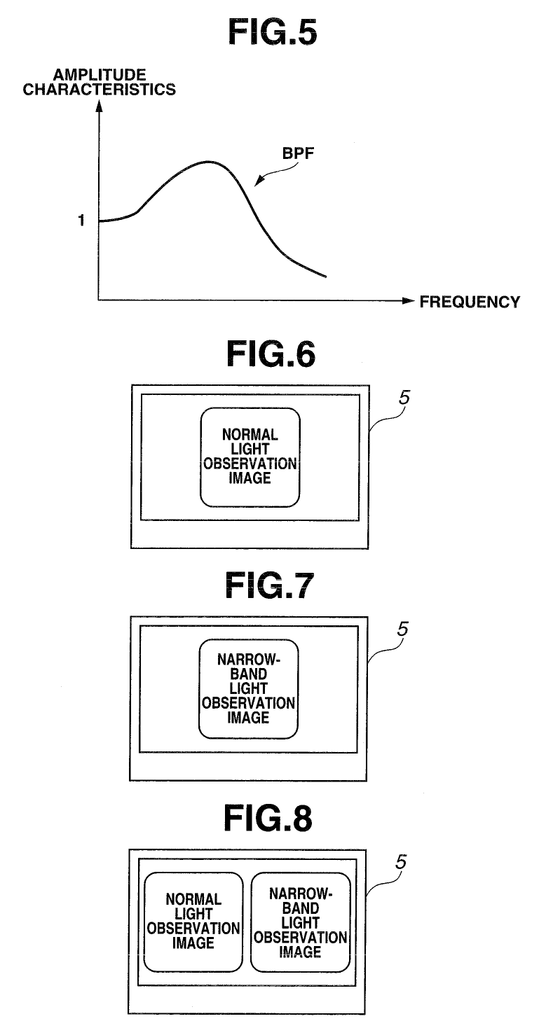

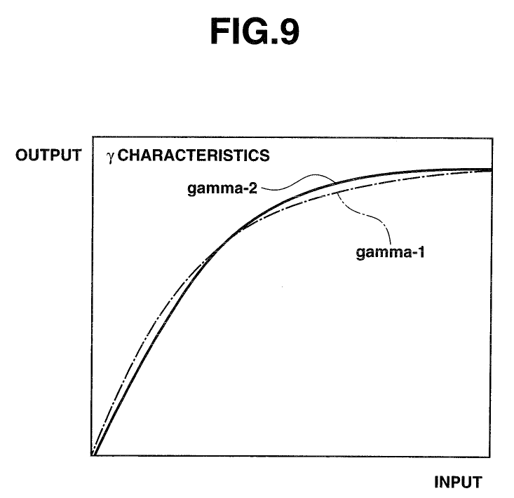

FIGS. 1 to 9 show a first embodiment of the present invention. FIG. 1 is a structural diagram showing a configuration of an endoscope apparatus. FIG. 2 is a structural diagram showing a configuration of a rotary filter shown in FIG. 1. FIG. 3 shows spectral characteristics of a filter set of the rotary filter shown in FIG. 2. FIG. 4 is a structural diagram showing a configuration of a respective band signal conversion section shown in FIG. 1. FIG. 5 shows amplitude characteristics of a BPF shown in FIG. 4. FIG. 6 is a first diagram showing a display example of an observation monitor shown in FIG. 1. FIG. 7 is a second diagram showing a display example of the observation monitor shown in FIG. 1. FIG. 8 is a third diagram showing a display example of the observation monitor shown in FIG. 1. FIG. 9 shows γ correction characteristics of a γ correction circuit shown in FIG. 1.

As shown in FIG. 1, an endoscope apparatus 1 of the present embodiment is made up of an electronic endoscope 3 which has a CCD 2 acting as biological image information acquiring means to be inserted into a body cavity to pick up an image of a tissue in the body cavity and acquire biological image information, a light source device 4 for supplying illumination light to the electronic endoscope 3, and a video processor 7 for performing signal processing on an image pickup signal from the CCD 2 of the electronic endoscope 3 and displaying an endoscope image on an observation monitor 5.

The light source device 4 includes a xenon lamp 11 acting as illuminating means for emitting illumination light (white light), a heat ray cut-off filter 12 for cutting off heat rays of white light, a beam limiting device 13 for controlling an amount of white light having passed through the heat ray cut-off filter 12, a rotary filter 14 acting as band limiting means for limiting illumination light to frame sequential light, a condenser lens 16 for condensing frame sequential light, which has passed through the rotary filter 14, on an incidence plane of a light guide 15 disposed in the electronic endoscope 3, and a control circuit 17 for controlling a rotation of the rotary filter 14.

As shown in FIG. 2, the rotary filter 14 is disc-shaped and has a rotation axis at a center of the filter. In a radiant part of the rotary filter 14, an R filter portion 14r, a G filter portion 14g, and a B filter portion 14b are disposed which compose the filter set for outputting frame sequential light having spectral characteristics shown in FIG. 3. The R filter portion 14r and the G filter portion 14g have overlapping spectral characteristics, and the spectral characteristics of the B filter portion 14b have a narrow band of 405 nm to 425 nm, for example, in a wave range of λ11 to λ12. The spectral characteristics may have a narrow band of 400 nm to 440 nm in the wave range of λ11 to λ12 of the B filter portion 14b.

As shown in FIG. 1, the rotary filter 14 is rotated by performing drive control on a rotary filter motor 18 through the control circuit 17.

The xenon lamp 11, the beam limiting device 13, and the rotary filter motor 18 are fed with power from a power supply section 10.

The video processor 7 includes a CCD drive circuit 20, an amplifier 22, a process circuit 23, an A/D converter 24, a white balance circuit (W.B) 25, a selector 100, a respective band signal conversion section 101 acting as biological image information converting means, a selector 102, a γ correction circuit 26, an expansion circuit 27, an emphasis circuit 28, a selector 29, synchronization memories 30, 31, and 32, an image processing circuit 33, D/A circuits 34, 35, and 36, a timing generator (T.G) 37, a control circuit 200, and a synthesis circuit 201 acting as display image generating means.

The CCD drive circuit 20 drives the CCD 2 provided in the electronic endoscope 3 and outputs a frame sequential image pickup signal synchronized with a rotation of the rotary filter 14. The amplifier 22 amplifies the frame sequential image pickup signal which has been obtained by picking up an image in a body cavity with the CCD 2 through an objective optical system 21 provided on an end of the electronic endoscope 3.

The process circuit 23 performs correlated dual sampling, noise removal, and the like on the frame sequential image pickup signal having passed through the amplifier 22. The A/D converter 24 converts the frame sequential image pickup signal, which has passed through the process circuit 23, to a digital frame sequential image signal.

The W.B 25 performs gain control and white balance processing on the frame sequential image signal, which has been digitized by the A/D converter 24, such that an R signal of the image signal and a B signal of the image signal have an equal brightness relative to a G signal of the image signal, for example (in other words, the W.B 25 obtains the R, G, and B signals when a subject has a white surface, for example, in a state in which a white cap is attached to the end of the electronic endoscope 3, and the W.B 25 multiplies the R signal and the B signal by a gain coefficient calculated based on a ratio of brightness relative to the G signal, so that white balance processing is performed so as to generate the R and B signals with a brightness equal to the brightness of the G signal).

The selector 100 outputs the frame sequential image signal from the W.B 25 dividedly to parts of the respective band signal conversion section 101. The respective band signal conversion section 101 converts the image signal from the selector 100, to a normal light observation image signal and a narrow-band light observation image signal. The selector 102 sequentially outputs the frame sequential image signals of the normal light observation image signal and the narrow-band light observation image signal from the respective band signal conversion section 101, to the γ correction circuit 26 and the synthesis circuit 201.

The γ correction circuit 26 performs γ correction on the frame sequential image signal from the selector 102 or the synthesis circuit 201. The expansion circuit 27 expands the frame sequential image signal having been subjected to γ correction by the γ correction circuit 26. The emphasis circuit 28 performs edge enhancement on the frame sequential image signal having been expanded by the expansion circuit 27. The selector 29 and the synchronization memories 30, 31, and 32 are provided to synchronize the frame sequential image signal from the emphasis circuit 28.

The image processing circuit 33 reads the frame sequential image signals stored in the synchronization memories 30, 31, and 32, and corrects a moving image color drift and so on. The D/A circuits 34, 35, and 36 convert the image signal from the image processing circuit 33 to analog video signals and output the signals to the observation monitor 5. The T.G 37 is fed with a sync signal, which has been synchronized with a rotation of the rotary filter 14, from the control circuit 17 of the light source device 4 and outputs various timing signals to the circuits in the video processor 7.

The electronic endoscope 2 further includes a mode switching switch 41 for feeding an output to a mode switching circuit 42 in the video processor 7. The mode switching circuit 42 of the video processor 7 outputs a control signal to a dimming control parameter switching circuit 44 and the control circuit 200. A dimming circuit 43 controls the beam limiting device 13 of the light source device 4 based on the dimming control parameter from the dimming control parameter switching circuit 44 and the image pickup signal having passed through the process circuit 23, so that a brightness is properly controlled.

Referring to FIG. 4, the respective band signal conversion section 101 will be described below. The selector 100 sequentially outputs the frame sequential image signals (respective color signals) from the W.B 25 to the respective band signal conversion section 101 based on the timing signals from the T.G 37.

As shown in FIG. 4, in the respective band signal conversion section 101, the R signal which is the color signal from the selector 100 is a wide-band R image signal suitable for a normal observation. The R signal is outputted through the respective band signal conversion section 101 to the selector 102 as a normal light observation R signal (hereinafter, will be referred to as WLI-R), and the R signal is outputted to a synchronization memory 110.

Further, in the respective band signal conversion section 101, the G signal which is the color signal from the selector 100 is a wide-band G image signal suitable for a normal observation. The G signal is passed through the respective band signal conversion section 101 and is outputted to the selector 102 as a normal light observation G signal (hereinafter, will be referred to as WLI-G), and the G signal is outputted to the synchronization memory 110 through a band-pass filter (BPF) 111. Since the G signal is passed through the BPF 111 having the amplitude characteristics of FIG. 5, a contrast is increased in tissue information on a deep portion reproduced by the wide-band G image signal and a high contrast image signal is generated which corresponds to an image obtained by irradiation with illumination light having spectral characteristics with a narrower band than illumination light having passed through the G filter portion 14g.

Moreover, in the respective band signal conversion section 101, the B signal which is the color signal from the selector 100 is outputted to the synchronization memory 110, is subjected to a predetermined brightness adjustment performed in a brightness adjustment circuit 113 through a low-pass filter (LPF) 112, and is outputted to the selector 102 as a normal light observation B signal (hereinafter, will be referred to as WLI-B). The B signal which is the color signal from the selector 100 is a narrow-band B image signal suitable for a narrow-band light observation. Since the B signal is passed through the LPF 112, a low-contrast image is generated which is equivalent to an image obtained by irradiation with illumination light having spectral characteristics with a wider band than illumination light having passed through the B filter portion 14b. Further, the B image signal is an image signal obtained by irradiation with narrow-band light on a blue short-wavelength side. Light is considerably absorbed by blood and so on and thus darkness increases. Therefore, the brightness adjustment circuit 113 is provided in the post-stage of the LPF 112 to adjust a brightness to a desired brightness and output the B signal as WLI-B to the selector 102.

The color signals inputted to the synchronization memory 101 are subjected to predetermined color conversion by a color conversion circuit 114 as expressed in formula (1) and are outputted to the selector 102 through a frame sequential circuit 115 as a frame sequential narrow-band light observation R signal (hereinafter, will be referred to as NBI-R), a frame sequential narrow-band light observation G signal (hereinafter, will be referred to as NBI-G) and a frame sequential narrow-band light observation B signal (hereinafter, will be referred to as NBI-B).

$$\begin{matrix}[ Formula 1 ] & \\ ( \begin{matrix}NBI - R \\ NBI - G \\ NBI - B\end{matrix} ) = ( \begin{matrix}0 & {m}_{1} & 0 \\ 0 & 0 & {m}_{2} \\ 0 & 0 & {m}_{3}\end{matrix} ) ( \begin{matrix}r \\ g \\ b\end{matrix} ) & ( 1 )\end{matrix}$$

where m1, m2, and m3 represent color conversion coefficients (real numbers) and r, g, and b represent color signals of R, G, and B which are inputted to the color conversion circuit 114.

The selector 102 outputs the frame sequential color signals of WLI-R, WLI-G, and WLI-B which compose the normal light observation image and the frame sequential color signals of NBI-R, NBI-G, and NBI-B which compose the narrow-band light image to the γ correction circuit 26 or the synthesis circuit 201 based on the control signal from the control circuit 200.

The image processing circuit 33 makes a moving image color drift correction to the color signals inputted from the synchronization memories 30, 31, and 32 and generates image signals to be outputted to the D/A circuits 34, 35, and 36. In other words, when the frame sequential color signals of WLI-R, WLI-G, and WLI-B are inputted, the image processing circuit 33 generates the normal light observation image. When the frame sequential color signals of NBI-R, NBI-G, and NBI-B are inputted, the image processing circuit 33 generates the narrow-band light image. When the image processing circuit 33 is fed with frame sequential color signals of a synthetic image signal which will be described later, the image processing circuit 33 generates a synthetic image signal having been subjected to a moving image color drift correction.

Further, as shown in FIGS. 6 and 7, the normal light observation image and the narrow-band light image are displayed on the observation monitor 5 while being switched in real time in a toggling manner in response to an operation of the mode switching switch 41. Moreover, as shown in FIG. 8, the normal light observation image and the narrow-band light image can be displayed in real time on the same screen of the observation monitor 5 in response to an operation of the mode switching switch 41.

In other words, in the present embodiment, the selector 102 is switched based on the control signal from the control circuit 200 to input two image signals of the same color signal (in the case of the R signal, WLI-R and NBI-R) to the synthesis circuit from memories (not shown) included in the selector 102, in a display mode for simultaneously displaying the normal light observation image and the narrow-band light observation image on the observation monitor 5.

The synthesis circuit 201 reduces the two inputted image signals and then synthesizes the image signals, so that a synthetic image signal is generated. The synthesis circuit 201 outputs the generated signal to the γ correction circuit 26 (the G and B signals are similarly synthesized, and WLI-R and NBI-R, WLI-G and NBI-G, and WLI-B and NBI-B are controlled based on the control signal from the control circuit 200, which will be described later, such that the signals are sequentially inputted to the synthesis circuit 201, the synthetic image signal being outputted from the synthesis circuit 201 to the γ correction circuit 26 in a frame sequential manner).

In a mode for displaying only one of the normal light observation image and the narrow-band light observation image, the selector 102 is not switched to output the image signals to the synthesis circuit 201 based on the control signal from the control circuit 200 but is switched to output the R signal, the G signal, and B signal of the normal light observation image or the narrow-band light observation image to the γ correction circuit 26 in a frame sequential manner.

The control circuit 200 identifies the mode based on a mode switching signal from the mode switching circuit 42 and switches the selector 102. After that, the control circuit 200 controls the R, G, and B signals in the selector 102 based on the timing signal from the T.G 37 such that the signals are sequentially outputted to the synthesis circuit 201 or the γ correction circuit 26 (when the signals are outputted to the synthesis circuit 201, WLI-R and NBI-R are simultaneously outputted, WLI-G and NBI-G are outputted at a next time, and then WLI-B and NBI-B are outputted at a subsequent time, which is repeatedly performed, and when the signals are outputted to the γ correction circuit 26, for example, in a mode for displaying the normal light observation image, WLI-R→WLI-G→WLI-B is repeated.).

The selector 102 includes the memories (not shown) in which WLI-R, WLI-G, WLI-B, NBI-R, NBI-G, and NBI-B inputted from the respective band signal converter 101 are stored based on the control signal from the control circuit 200 only in the mode for simultaneously displaying the normal light observation image and the narrow-band light image.

In the above explanation, the synthesis circuit 201 reduces and synthesizes the two image signals so as to laterally place the image signals. The synthesis circuit 201 may synthesize the image signals by detecting only subject image signals in the image signals (image signal portions based on a subject image, the image signals corresponding to the normal light observation image other than a margin in FIG. 6) and laterally placing only the subject image signals having been detected from the two image signals.

In the present embodiment, as shown in FIG. 9, the γ correction circuit 26 uses different γ correction characteristics between WL-R, WLI-G and WLI-B and NBI-R, NBI-G and NBI-B which are the frame sequential signals outputted from the selector 102. In other words, gamma-1 characteristics of FIG. 9 are used for the frame sequential color signals of WLI-R, WLI-G, and WLI-B which compose the normal light observation image, and gamma-2 characteristics of FIG. 9 are used for NBI-R, NBI-G, and NBI-B which compose the narrow-band light image, in order to achieve a high contrast.

In other words, in the case of the mode for displaying only one of the normal light observation image and the narrow-band light observation image, the γ correction circuit 26 is fed with the control signal (the display mode for displaying only one of the normal light observation image and the narrow-band light observation image has been identified) from the control circuit 200.

As shown in FIG. 9, in the mode for displaying the normal light observation image, the γ correction circuit 26 makes a γ correction according to the gamma-1 characteristics based on the control signal. In a mode for displaying the narrow-band light observation image, the γ correction circuit 26 makes a γ correction according to the gamma-2 characteristics (In this case, the γ correction circuit 26 does not identify the image signal based on the control signal which will be described later).

On the other hand, in the mode for simultaneously displaying the normal light observation image and the narrow-band light observation image, the γ correction circuit 26 is fed with a sync signal outputted from the synthesis circuit 201 and is fed with the control signal (the simultaneous display mode has been identified) from the control circuit 200.

The γ correction circuit 26 identifies, as shown in FIG. 9, the WLI image signal and the NBI image signal based on the control signal and uses the gamma-1 characteristics for the WLI image signal and the gamma-2 characteristics for the NBI image signal. For the identification of the image signals, image region information is used. For example, in the display of FIG. 8, the image signal corresponding to a left half of the screen is identified as the WLI image signal and the gamma-1 characteristics are used. The image signal corresponding to a right half is identified as the NBI image signal and the gamma-2 characteristics are used.

As described above, in the present embodiment, the respective band signal conversion section 101 generates WLI-R, WLI-G, and WLI-B for generating the normal light observation image and NBI-R, NBI-G, and NBI-B for generating the narrow-band light image, based on the RGB signals obtained by irradiation with frame sequential light of a set of the rotary filter 14. In other words, by irradiation with frame sequential light through the rotary filter 14 made up of the set of the R filter portion 14r, the G filter portion 14g, and the B filter portion 14b, the normal light observation image and the narrow-band light image can be generated in real time. Thus it is possible to simplify the configuration of the apparatus and simultaneously observe the normal light observation image and the narrow-band light image.

The synthesis circuit 201 synthesizes the normal light observation image and the narrow-band light image, so that the normal light observation image and the narrow-band light image can be simultaneously observed.

### Second Embodiment

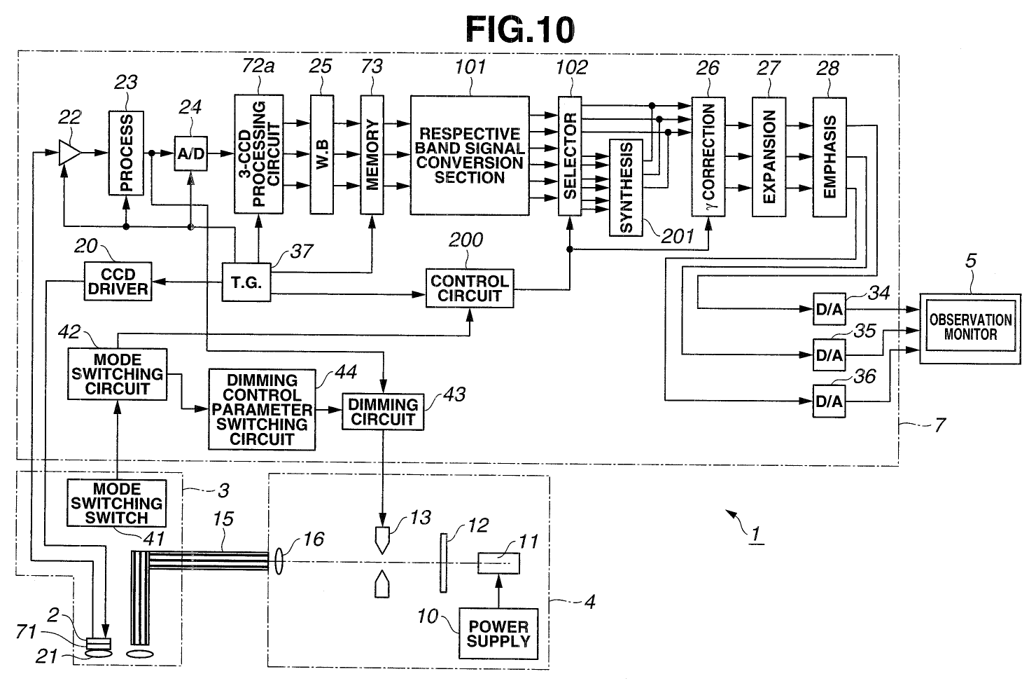

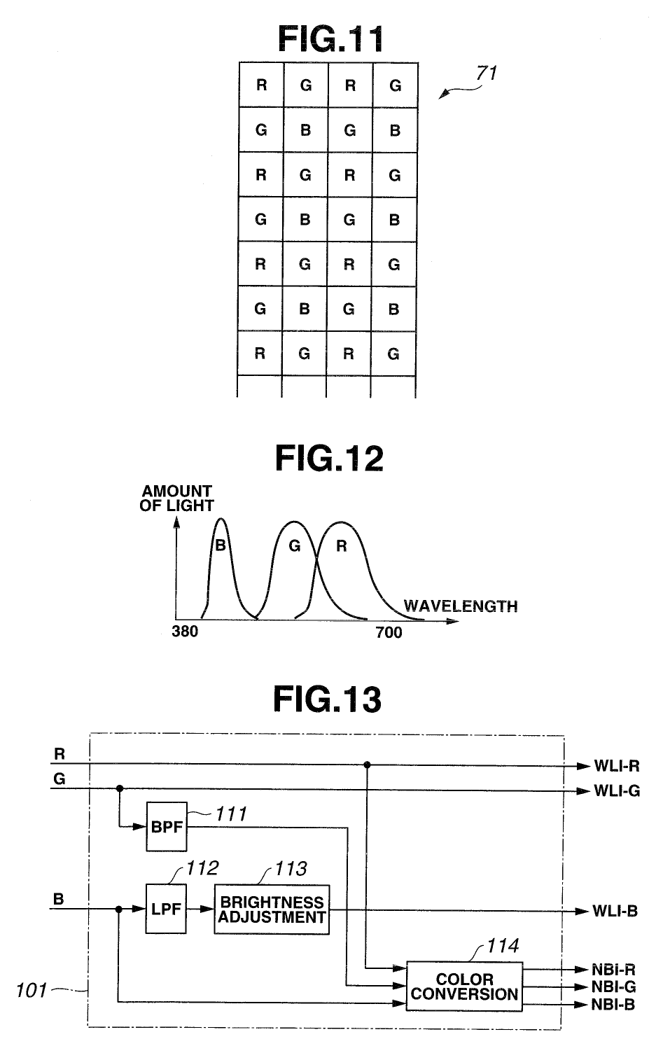

FIGS. 10 to 13 show a second embodiment of the present invention. FIG. 10 is a structural diagram showing a configuration of an endoscope apparatus. FIG. 11 is a structural diagram showing a configuration of a primary color filter shown in FIG. 10. FIG. 12 shows a transmission property of the primary color filter shown in FIG. 11. FIG. 13 is a structural diagram showing a configuration of a respective band signal conversion section shown in FIG. 10.

The second embodiment is substantially the same as the first embodiment and thus only different points will be described below. The same configurations as in the first embodiment will be indicated by the same reference numerals and the explanation thereof is omitted.

In the first embodiment, the normal light observation image and the narrow-band light image are generated by frame sequential image pickup observation through the rotary filter 14. In the present embodiment, as shown in FIG. 10, white light is applied to a tissue in a body cavity and is separated into colors through a primary color filter 71, and a normal light observation image and a narrow-band light image are generated through simultaneous-type image pickup observation in which an image is picked up by a CCD 2. FIG. 11 shows the configuration of the primary color filter 71, and FIG. 12 shows the transmission property of each color filter.

In a video processor 7 of the present embodiment, as shown in FIG. 10, an RGB image signal, which is a single-CCD (single color/pixel) image signal from an A/D converter 24, is subjected to 3-CCD processing (three RGB colors/pixel) into an R signal, a G signal, and a B signal in a 3-CCD processing circuit 72a. Further, the R signal, the G signal, and the B signal which have been subjected to 3-CCD processing in the 3-CCD processing circuit 72a are subjected to white balance processing by a W.B 25 as in the first embodiment. After that, the R signal, the G signal, and the B signal which have been subjected to white balance processing are temporarily stored in a memory 73, and then the R signal, the G signal, and the B signal are read from the memory 73 and are outputted to a respective signal conversion section 101.

The respective signal conversion section 101 is configured substantially as in the first embodiment. As shown in FIG. 13, in the respective band signal conversion section 101 of the present embodiment, the R signal picked up through the primary color filter 71 is a wide-band R image signal suitable for a normal observation (see FIG. 12), and the R signal is outputted to a selector 102 as WLI-R through the respective band signal conversion section 101 and is outputted to a color conversion circuit 114. Further, the G signal picked up through the primary color filter 71 is a wide-band G image signal suitable for a normal observation (see FIG. 12), and the G signal is outputted to the selector 102 as WLI-G through the respective band signal conversion section 101 and is outputted to the color conversion circuit 114 through a BPF 111. Moreover, the B signal picked up through the primary color filter 71 is a narrow-band B image signal suitable for a narrow-band light observation (see FIG. 12). The B signal is outputted to the color conversion circuit 114 and a brightness is adjusted by a brightness adjustment circuit 113 through a LPF 112, and the B signal is outputted to the selector 102 as WLI-B.

The color conversion circuit 114 performs predetermined color conversion on the inputted image signals and outputs the signals to the selector 102 as NBI-R, NBI-G, and NBI-B.

After that, the selector 102 outputs WLI-R, WLI-G, WLI-B, and NBI-R, NBI-G, and NBI-B to a γ correction circuit 26 or a synthesis circuit 201 based on a control signal from a control circuit 200. The synthesis circuit 201 synthesizes the inputted image signals.

In other words, in the present embodiment, the selector 102 is switched to input the six image signals (WLI-R, WLI-G, WLI-B, NBI-R, NBI-G, and NBI-B) to the synthesis circuit 201 from memories (not shown) included in the selector 102, in a display mode for simultaneously displaying the normal light observation image and the narrow-band light observation image on an observation monitor 5.

The synthesis circuit 201 reduces the two image signals of the same color (WLI-R and NBI-R, WLI-G and NBI-G, and WLI-B and NBI-B) and then synthesizes the image signals, so that a synthetic image signal (RGB image signal) is generated. The synthetic image signal is outputted to the γ correction circuit 26.

In a mode for displaying only one of the normal light observation image and the narrow-band light observation image, the selector 102 is not switched to output the image signals to the synthesis circuit 201 based on the control signal from the control circuit 200 but is switched to output the R signal, the G signal, and B signal of the normal light observation image or the narrow-band light observation image to the γ correction circuit 26.

The control circuit 200 identifies the mode based on a mode switching signal from a mode switching circuit 42 and switches the selector 102. After that, the control circuit 200 controls the R, G, and B signals in the selector 102 based on a timing signal from a T.G 37 such that the signals are outputted to the synthesis circuit 201 or the γ correction circuit 26 (when the signals are outputted to the synthesis circuit 201, WLI-R, WLI-G, WLI-B, NBI-R, NBI-G, and NBI-B are simultaneously outputted, and when the signals are outputted to the γ correction circuit 26, for example, in a mode for displaying the normal light observation image, WLI-R, WLI-G, and WLI-B are controlled to be simultaneously outputted from the selector 102).

In the above explanation, the synthesis circuit 201 reduces and synthesizes the two image signals of the same color signal so as to laterally place the image signals. The synthesis circuit 201 may synthesize the image signals by detecting only subject image signals in the image signals (image signal portions based on a subject image, the image signals corresponding to the normal light observation image other than a margin in FIG. 8) and laterally placing only the subject image signals having been detected from the two image signals.

The γ correction circuit 26 identifies, as in the first embodiment, the WLI image signal and the NBI image signal based on the control signal and uses gamma-1 characteristics for the WLI image signal and gamma-2 characteristics for the NBI image signal. For the identification of the image signals, image region information is used. For example, in display of FIG. 8, the image signal corresponding to a left half of a screen is identified as the WLI image signal and the gamma-1 characteristics are used. The image signal corresponding to a right half is identified as the NBI image signal and the gamma-2 characteristics are used.

In the case of the mode for displaying only one of the normal light observation image and the narrow-band light observation image, the γ correction circuit 26 makes a γ correction to the normal light observation image according to the gamma-1 characteristics based on the control signal from the control signal, and makes a γ correction to the narrow-band light observation image according to the gamma-2 characteristics (in this case, the γ correction circuit 26 does not identify the image signal based on the control signal).

The video processor 7 of the present embodiment includes, as in the first embodiment, the γ correction circuit 26 for making a γ correction to the image signals having passed through the selector 102, an expansion circuit 27 for expanding the image signals having been subjected to the γ correction, and an emphasis circuit 28 for performing edge enhancement on the expanded image signals. The image signals from the emphasis circuit 28 are converted to analog video signals by D/A circuits 34, 35, and 36 and are outputted to the observation monitor 5.

In the present embodiment, as shown in FIG. 10, the control circuit 200 is provided. The control circuit 200 is fed with a CCD driving signal from a CCD driver 20. The control circuit 200 detects image pickup of one frame based on the CCD driving signal from the CCD driver 20, controls the selector 102, and outputs WLI-R, WLI-G, WLI-B, NBI-R, NBI-G, and NBI-B from the selector 102 to the γ correction circuit 26 or the synthesis circuit 20.

Thus the present embodiment can achieve the same effect as in the first embodiment.

### Third Embodiment

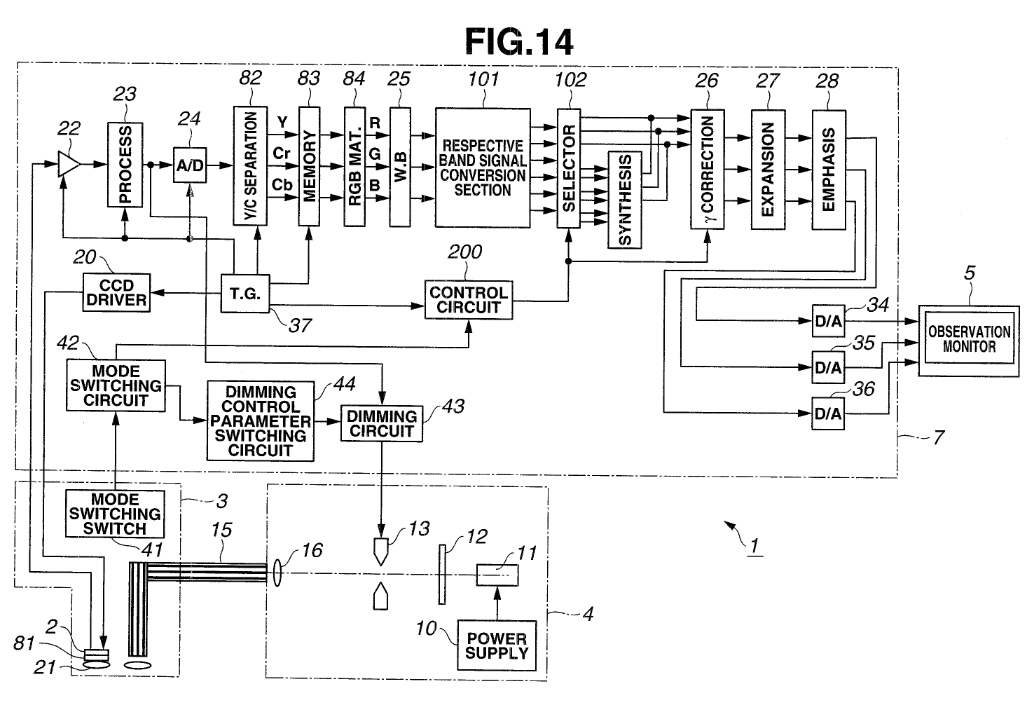

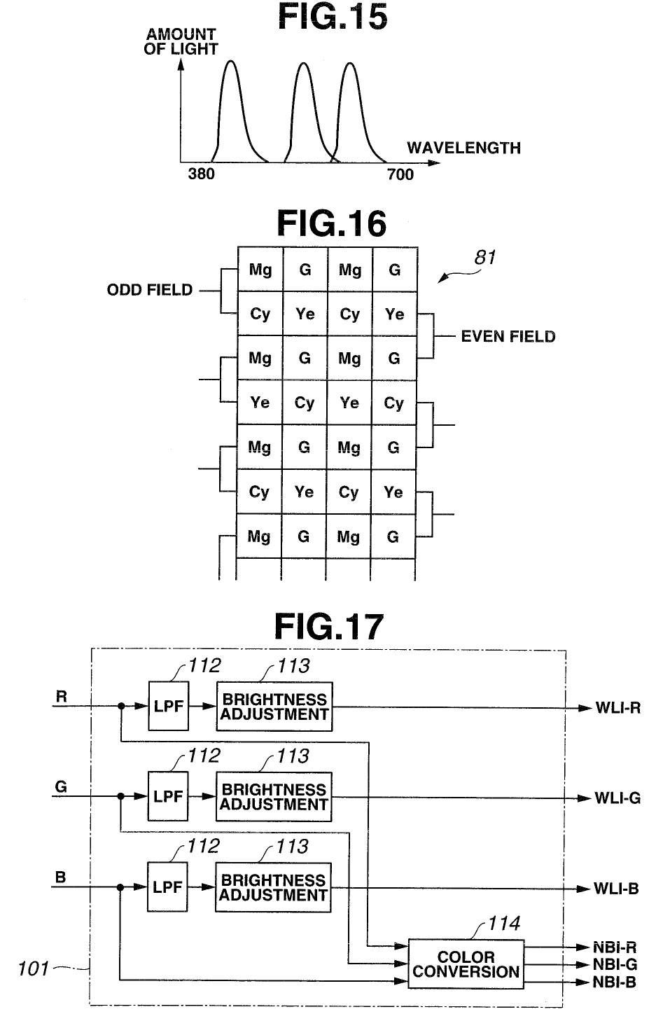

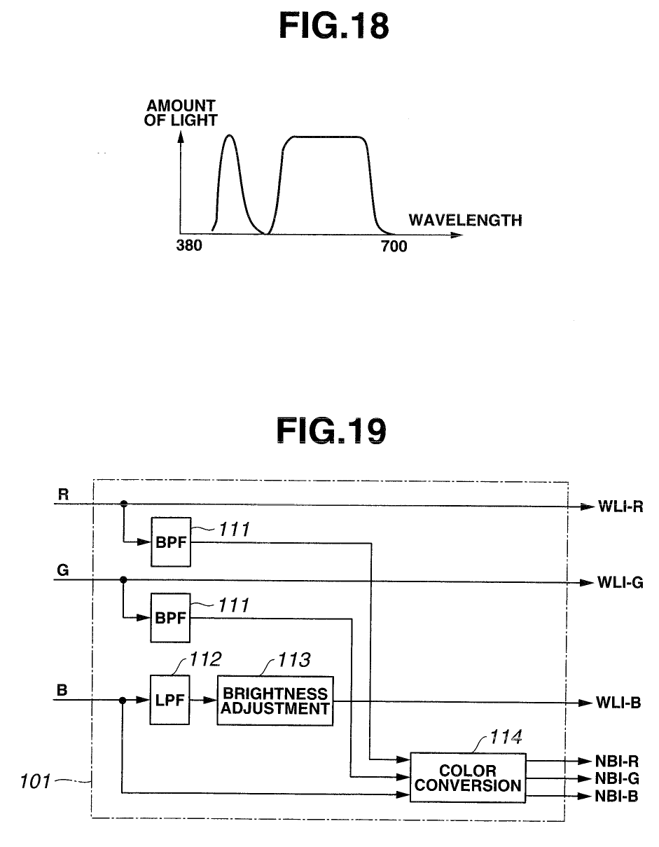

FIGS. 14 to 19 show a third embodiment of the present invention. FIG. 14 is a structural diagram showing a configuration of an endoscope apparatus. FIG. 15 shows a transmission property of a heat ray cut-off filter shown in FIG. 14. FIG. 16 is a structural diagram showing a configuration of a complementary color filter shown in FIG. 14. FIG. 17 is a structural diagram showing a configuration of a respective band signal conversion section shown in FIG. 14. FIG. 18 shows a modification of a transmission property of the heat ray cut-off filter shown in FIG. 14. FIG. 19 is a structural diagram showing a configuration of a modification of a respective band signal conversion section shown in FIG. 14.

The third embodiment is substantially the same as the second embodiment and thus only different points will be described below. The same configurations as in the second embodiment will be indicated by the same reference numerals and the explanation thereof is omitted.

In the present embodiment, as shown in FIG. 14, a light source device 4 is substantially the same as in the second embodiment and a heat ray cut-off filter 12 has the transmission property of FIG. 15. Further, a complementary color filter 81 configured as shown in FIG. 16 is provided on an image pickup surface of a CCD 2, instead of a primary color filter 71.

In a video processor 7 of the present embodiment, as shown in FIG. 14, an image signal from an A/D converter 24 is subjected to Y/C separation (separated into luminance/color difference signals) in a Y/C separation circuit 82. A luminance signal Y and color difference signals Cr and Cb which have been subjected to Y/C separation are temporarily stored in a memory 83, and then the luminance signal Y and the color difference signals Cr and Cb are read from the memory 83 and are converted to RGB signals in an RGB matrix circuit 84. The R signal, the G signal, and the B signal from the RGB matrix circuit 84 are subjected to white balance processing by a W.B 25 as in the first embodiment. After that, the R signal, the G signal, and the B signal which have been subjected to white balance processing are outputted to a respective band signal conversion section 101. Configurations following the respective band signal conversion section 101 are similar to the configurations of the second embodiment.

The transmission property of the heat ray cut-off filter 12 serving as band limiting means is narrow-band characteristics as shown in FIG. 15. Thus as shown in FIG. 17, the respective band signal conversion section 101 of the present embodiment performs predetermined color conversion on the R signal, the G signal, and the B signal in a color conversion circuit 114, and then outputs the signals to a selector 102 as NBI-R, NBI-G, and NBI-B. Further, the respective band signal conversion section 101 adjusts a brightness for each of the R signal, the G signal, and the B signal in brightness adjustment circuits 113 through LPFs 112 and outputs the signals to the selector 102 as WLI-R, WLI-G, and WLI-B.

Thus the present embodiment can achieve the same effect as in the second embodiment.

The transmission property of the heat ray cut-off filter 12 is not limited to the property of FIG. 15 and the heat ray cut-off filter 12 may have a transmission property of FIG. 18. In this case, as shown in FIG. 19, the R signal and the G signal are outputted as WLI-R and WLI-G to the selector 102 through the respective band signal conversion section 101 of the present embodiment. The B signal is subjected to brightness adjustment in the brightness adjustment circuit 113 through the LPF 112 and is outputted to the selector 102 as WLI-B. Further, the R signal and the G signal are outputted to the color conversion circuit 114 through BPFs 111, are subjected to the predetermined color conversion with the B signal in the color conversion circuit 114, and then are outputted to the selector 102 as NBI-R, NBI-G, and NBI-B.

The present invention is not limited to the foregoing embodiments and various changes and modifications can be made without changing the subject matter of the present invention.

What is claimed is:

1. An endoscope apparatus, comprising:
an illuminating unit for applying illumination light to a subject;
a biological image information acquiring unit for receiving a subject image of the subject having been irradiated with the illumination light from the illuminating unit, and obtaining biological image information of the subject;
a band limiting unit which is disposed on an optical path from the illuminating unit to the biological image information acquiring unit and limits, to a predetermined bandwidth, at least one of a plurality of wavelength bands allocated according to penetration depths of light in the subject;
a biological image information converting section for converting the biological image information obtained by the biological image information acquiring unit, to first biological image signal information corresponding to irradiation with band limited light of the plurality of wavelength bands with the predetermined bandwidth and second biological image information corresponding to irradiation with the illumination light; and
a display image generating unit for generating a display image to be displayed on a display unit, based on the first biological image signal information and the second biological image signal information which have been converted by the biological image information converting section.

2. The endoscope apparatus according to claim 1, wherein the band limiting unit limits, to the predetermined bandwidth, the wavelength band of the illumination light from the illuminating unit.

3. The endoscope apparatus according to claim 1, wherein the band limiting unit limits, to the predetermined bandwidth, the wavelength band of the subject image received by the biological image information acquiring unit.

4. The endoscope apparatus according to claim 2, wherein the illumination light is RGB frame sequential light.

5. The endoscope apparatus according to claim 2, wherein the illumination light is white light,
the biological image information acquiring unit is a CCD, and
the band limiting unit is a primary color filter disposed on an image pickup surface of the CCD.

6. The endoscope apparatus according to claim 3, wherein the illumination light is white light,
the biological image information acquiring unit is a CCD, and
the band limiting unit is a primary color filter disposed on an image pickup surface of the CCD.

7. The endoscope apparatus according to claim 2, wherein the biological image information acquiring unit is a CCD having a complementary color filter disposed on an image pickup surface of the CCD.

8. The endoscope apparatus according to claim 1, wherein the biological image converting section has an image signal converting section for performing image signal conversion differently between the first biological image information and the second biological image information.

9. The endoscope apparatus according to claim 8, wherein the image signal converting section is made up of a contrast changing unit for changing a contrast of an image signal and/or a color converting section for converting a color of the image signal.

10. A signal processing method of an endoscope apparatus, comprising:
an illuminating step of applying illumination light to a subject;
a biological image information acquiring step of receiving a subject image of the subject having been irradiated with the illumination light, and obtaining biological image information of the subject;
a band limiting step of limiting, to a predetermined bandwidth, at least one of a plurality of wavelength bands allocated according to penetration depths of light in the subject, on an optical path from the illuminating unit to the biological image information acquiring unit;
a biological image information converting step of converting the biological image information obtained in the biological image information acquiring step, to first biological image signal information corresponding to irradiation with band limited light of the plurality of wavelength bands with the predetermined bandwidth and second biological image information corresponding to irradiation with the illumination light; and
a display image generating step of generating a display image to be displayed on a display unit, based on the first biological image signal information and the second biological image signal information which have been converted in the biological image information converting step.

11. The signal processing method of the endoscope apparatus according to claim 10, wherein in the band limiting step, the wavelength band of the illumination light from the illuminating unit is limited to the predetermined bandwidth.

12. The signal processing method of the endoscope apparatus according to claim 10, wherein in the band limiting step, the wavelength band of the subject image received by the biological image information acquiring unit is limited to the predetermined bandwidth.

13. The signal processing method of the endoscope apparatus according to claim 11, wherein the illumination light is RGB frame sequential light.

14. The signal processing method of the endoscope apparatus according to claim 11, wherein the illumination light is white light,
the biological image information acquiring step is an image pickup step performed by a CCD, and
the band limiting step is a band limiting step performed by a primary color filter disposed on an image pickup surface of the CCD.

15. The signal processing method of the endoscope apparatus according to claim 12, wherein the illumination light is white light,
the biological image information acquiring step is an image pickup step performed by a CCD, and
the band limiting step is a band limiting step performed by a primary color filter disposed on an image pickup surface of the CCD.

16. The signal processing method of the endoscope apparatus according to claim 11, wherein the biological image information acquiring step is an image pickup step performed by a CCD having a complementary color filter disposed on an image pickup surface of the CCD.

17. The signal processing method of the endoscope apparatus according to claim 10, wherein the biological image converting step has an image signal converting step of performing image signal conversion differently between the first biological image information and the second biological image information.

18. The signal processing method of the endoscope apparatus according to claim 17, wherein the image signal converting step is made up of a contrast changing step of changing a contrast of an image signal and/or a color converting step of converting a color of the image signal.
